(19) United States
(12) Patent Application Publication
(10) Pub. No.: US 2003/0138345 A1
(43) Pub. Date: Jul. 24, 2003

(54) Postage stamp indicating completion of sterilization process

(76) Inventor: Nikolaus Z. Schwabe, Dallas, TX (US)

Correspondence Address: Michael L. Diaz, Michael L. Diaz, P.C., Suite 200, 555 Republic Drive, Plano, TX, 75074, US

(21) Appl. No.: 10/051,641

(22) Filed: Jan. 18, 2002

(51) Int. Cl. A61L 2/00

(52) U.S. Cl. 422/22; 422/1; 340/540

(57) ABSTRACT

A postage stamp providing an indication of successful completion of a sterilization process. The postage stamp is affixed to a mail article. The mail article is then collected by the postal service for processing. The mail article is subjected to sterilization by a sterilization apparatus to sterilize the mail article. Upon being successfully sterilized, the postage stamp turns color to indicate completion of the sterilization process.

### BACKGROUND OF THE INVENTION

1. Technical Field of the Invention

This invention relates to postage stamps and, more particularly, to a postage stamp providing an indication that a sterilization process has been successfully conducted upon the stamp and its associated letter or package.

2. Description of Related Art

Terrorist activities have started to creep into many aspects of our society which were once thought to be completely safe. Unfortunately, in several incidences, terrorists have initiated delivery of harmful microorganisms to people through letters and packages delivered through conventional postal methods. For example, recent activities have shown terrorists have attempted the transportation of anthrax spores via letters delivered by the United States Postal Service. These infected letters have caused great harm to many people and businesses, both physically and economically. Due to the threat of anthrax spores being transported by the U.S. Mail in letters and packages, the U.S. Postal Service has experienced a significant decrease (approximately thirty percent) in the use of mail service. To combat this new form of terrorism, the United States Postal Services has initiated sterilization processes which sterilize postal letters and packages prior to delivery. However, large logistical problems must be overcome to make the sterilization process effective. Currently, the public has a general fear of receiving mail from unknown sources. The public's fear will be diminished once they are confident that the mail is properly sterilized. Although the United States Postal Service plans on sterilizing all mail, there is certainly the possibility that some mail will inadvertently circumvent the sterilization process. The task of sterilizing all pieces of mail is daunting, considering the volumes of mail that are delivered each day. In addition, because of this possibility of the mail circumventing the sterilization process, the public will never be totally confident that their mail is safe without receiving some indication that the mail has been sterilized. An apparatus and method are needed which clearly, efficiently, and effectively indicates to the general public, as well as postal workers, that specific pieces of mail have undergone the sterilization process. In addition, the method must minimize the expense and labor required to alter the present postal processing of mail.

Although there are no known prior art teachings of a solution to the aforementioned deficiency and shortcoming such as that disclosed herein, prior art references that discuss subject matter that bears some relation to matters discussed herein are U.S. Pat. No. 4,015,937 to Miyamoto et al. (Miyamoto), U.S. Pat. No. 5,206,118 to Sidney et al. (Sidney), U.S. Pat. No. 5,451,792 to Maguire et al. (Maguire), and U.S. Pat. No. 6,114,707 to Willems et al. (Willems).

Miyamoto discloses a process for detecting the completion of a gaseous alkaline oxide sterilization treatment of medical or surgical equipment by the color change of particular dye indicators. Although Miyamoto discloses a process for indicating the completion of a sterilization process, Miyamoto does not teach or suggest a device which provides an indication of sterilization of a mail article. In addition, Miyamoto does not teach or suggest utilizing a postage stamp to provide this indication.

Sidney discloses a color-change dosimeter film made of a polymer which is dispersed as an acid-sensitive dye. The dye is substantially free from groups that are sensitive to high-energy radiation and becomes colored in acid. This color-change dosimeter film remains substantially colorless upon exposure to artificial illumination. The dosimeter becomes colored upon exposure to high-energy radiation and accurately indicates small changes in dosage by the intensity of its color and remains substantially unchanged in its intensity of that color after being stored for several months. Sidney does not teach or suggest utilizing the dosimeter as a postage stamp on an article to be mailed. Sidney merely discloses an improved dosimeter for indicating exposure to radiation.

Maguire discloses a gamma radiation dosimeter having a polyester film substrate with a central circular region. The central region is coated with a gamma-radiation sensitive layer which is covered by an overlayer. The central region is surrounded an annular region which is divided into six equally-sized sections, each of which is printed in a color which corresponds to the color developed in the central regions when it has been exposed to successive increasing amounts of gamma radiation. After exposure of the device, the amount of radiation that the device has been exposed can be determined by comparing the color generated in the region with the colors of the six sections. The color of the section most closely corresponding to that of the central region indicates the dose of gamma radiation which the device has been exposed. Although Maguire discloses a dosimeter providing an indication of exposure to radiation, Maguire does not teach or suggest an indicator affixed to a postage stamp.

Willems discloses a method for checking whether an article has been inspected by penetrating radiation. The article is provided with a heat stimulable storage phosphor in which penetrating radiation leaves a human or machine-readable semipermanent or permanent mark. The article is irradiated in order that both the article and the stimulable storage phosphor are irradiated. The article is checked by heating the storage phosphor, releasing flourescent light and detecting the flourescent light by human eye. However, Willems does not teach or suggest an indicator used as a postage stamp for indicating successful completion of a sterilization process.

Review of the foregoing references reveals no disclosure or suggestion of an apparatus, system or method for providing an efficient and simple indication to the public and postal workers that a piece of mail has been properly sterilized. It is an object of the present invention to provide such an apparatus, system, and method.

### SUMMARY OF THE INVENTION

In one aspect, the present invention is a system for indicating successful sterilization of a mail article. The system includes a mail article and a postage stamp affixed to the mail article. The postage stamp has a color indicator showing a first color. In addition, the system includes a sterilization apparatus conducting a sterilization process for sterilizing a plurality of mail articles. When the mail article is subjected to the sterilization process by the sterilization apparatus, the color indicator changes from the first color to a second color to indicator successful sterilization of the mail article.

In another aspect, the present invention is a method of indicating successful completion of sterilization on a mail article by a sterilization apparatus. The method begins by a sender affixing a postage stamp to the mail article. Next, the mail article is collected by a postal service. The mail article is then subjected to a sterilization process by the sterilization apparatus. A portion of the postage stamp changes from a first color to a second color upon successful completion of the sterilization process. The mail article is then delivered to a specified recipient.

In still another aspect, the present invention is a stamp for indicating proper sterilization of a mail article. The stamp includes a sheet of material having an outer surface and a back surface. The outer surface displays a color indicator. The back surface has an adhesive for affixing the sheet of material to the mail article. The color indicator indicates a first color prior to being subjected to a sterilization process and indicating a second color after being subjected to a sterilization process.

### BRIEF DESCRIPTION OF THE DRAWINGS

The invention will be better understood and its numerous objects and advantages will become more apparent to those skilled in the art by reference to the following drawings, in conjunction with the accompanying specification, in which:

FIG. 1 is a simplified block diagram illustrating the components of a postal system in the preferred embodiment of the present invention;

FIG. 2 is a front view of the mailing article of FIG. 1;

FIG. 3 is an enlarged front view of the postage stamp of FIG. 2;

FIG. 4 is a flow chart outlining the steps for processing a plurality of mail articles according to the teachings of the present invention; and

FIG. 5 is a front view of an envelop having a metering mark in an alternate embodiment of the present invention.

### DETAILED DESCRIPTION OF EMBODIMENTS

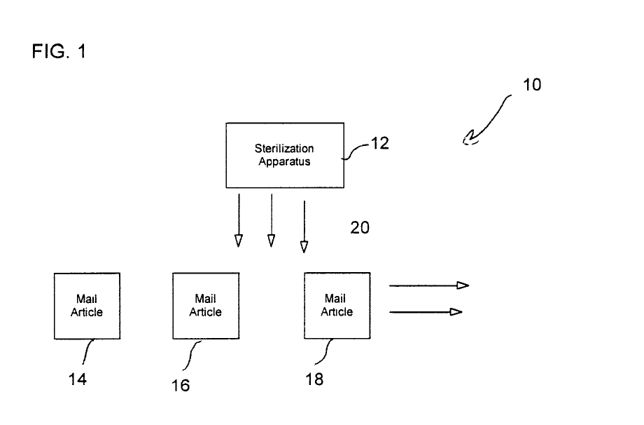

The present invention is a postage stamp which indicates exposure to radiation. FIG. 1 is a simplified block diagram illustrating the components of a postal system 10 in the preferred embodiment of the present invention. The system includes a sterilization apparatus 12 and a plurality of mailing articles 14, 16, and 18. During the processing of the mailing articles, the sterilization apparatus emits electronic beams 20 to sterilize the mailing articles, thus killing any harmful microorganisms. Sterilization devices are well known in the packaging industry. Any sterilization device may be utilized. In the preferred embodiment of the present invention, the sterilization apparatus employs an electron beam to kill the harmful microorganisms. However, in other embodiments, the sterilization apparatus utilizes radiation, such as gamma radiation to sterilize the mail articles. In addition, the sterilization apparatus may emit a gas, such as ethylene oxide, for sterilizing the mail articles.

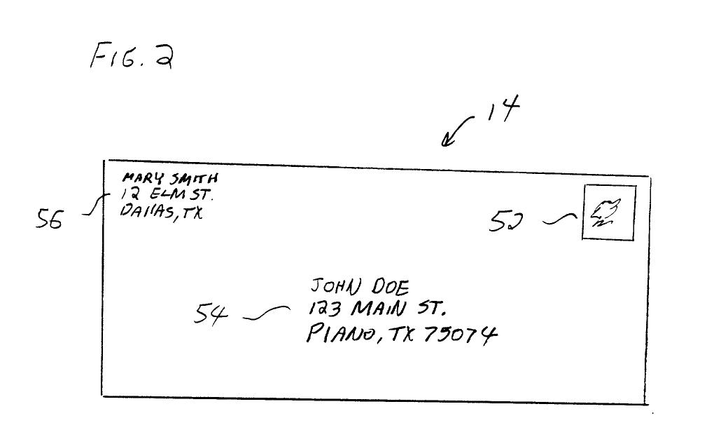

FIG. 2 is a front view of the mailing article 14 of FIG. 1. The mailing article may be any piece of mail, such as a letter, package, or postcard. As illustrated, the mailing article is a conventional envelop. The envelop includes a postage stamp 52. Additionally, the envelop includes an address section 54 and a return address section 56.

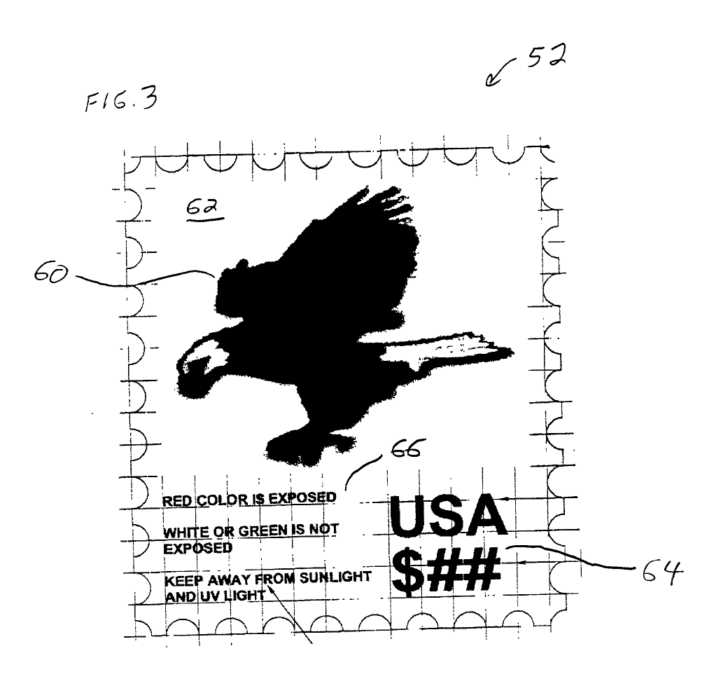

FIG. 3 is an enlarged front view of the postage stamp 52 of FIG. 2. The postage stamp includes a design section 60 positioned on a background 62. In addition, the postage stamp includes a monetary value section 64 and an optional explanation section 66. The postage stamp is a conventional postage stamp having an adhesive substance affixed to a back surface. The adhesive may requiring moistening or allow direct attachment on the envelop without moistening the back surface of the stamp.

The design section typically depicts an illustration. The background section 62 may include an illustration, solid color or remain white. In the preferred embodiment of the present invention, both the design section 60 and the background section 62 include an ink which changes color when exposed to an electronic beam. The transformation of the color of on a label is well known to those skilled in the art of sterilization, such as those discussed in the prior art references above.

For example, prior to being exposed to an electron beam, the design section 60 and the background section 62 both may show a green or white color. When the postage stamp is sufficiently exposed to the electron beam from the sterilization apparatus 12, the background section 62 and the design section 60 transform to a different color, such as red.

The stamp, as typical in existing postage stamp, also provides an indication of the monetary value of the stamp, as illustrated by the monetary value section 64. Additionally, an explanation section 66 providing text explaining the color coding of the stamp may also be optionally displayed.

Although the preferred embodiment of the present invention provides an indication of exposure to an electron beam, it should be understood that color transformation of the stamp may occur for exposure to any type of sterilization used by the sterilization apparatus 12, and not limited to an electron beam. Additionally, only a portion of the stamp, rather than both the design section and background section of the stamp, may indicate exposure to the electron beam. Thus, a small portion of the stamp may provide a color indication when exposed to the sterilization process.

With reference to FIGS. 1-3, the operation of the system 10 will now be explained. The plurality of mail articles 14, 16, and 18 are affixed with a postage stamp 52 of a specific monetary value, as indicated by the monetary value section 64 located on each postage stamp 52. Each sender then sends through the postal system, each mail article. The postal service collects the mail articles from each sender. The mail articles are then processed at a postal facility. The normal processing of the mail may include sorting, affixing a post mark indicating payment of mailing fees and date of receipt of the mail. Either before or after this processing of the mail, the mail article is exposed to electron beam from the sterilization apparatus 12.

The sterilization apparatus emits an electron beam sufficient to kill any harmful microorganisms. The electron beam also contacts the postage stamp 52. The design section 60 and background section 62, upon being exposed to the electron beam from the sterilization apparatus 12, changes color. As illustrated, the background section 62 and design section transforms color (e.g., green/white to red).

The mail articles 14, 16, 18 are then delivered to each specified addressee. The addressee and postal employees all are provided with an indication that each mail article has been properly sterilized, thus indicating that the mail articles are safe to handle or open. If the postage stamp does not show a transformation of color, a readily apparent indication is displayed to all those handling the mail articles that the mail article has not been properly sterilized. In such a situation, limited handling of the mail article may be required. If a postal employee notices that the postage stamp is not indicating completion of sterilization process, the mail may be properly processed through the sterilization apparatus.

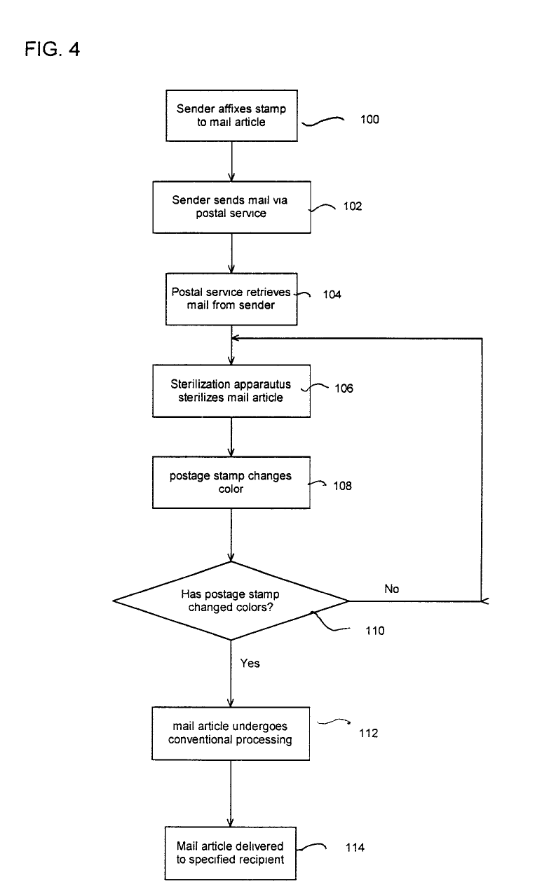

FIG. 4 is a flow chart outlining the steps for processing a plurality of mail articles according to the teachings of the present invention. With reference to FIGS. 1-3, 4A and 4B, the steps of the method will now be explained. The method begins in step 100 where a mail sender affixes a postage stamp 52 to the mail article 14. Next, in step 102, the sender sends the mail article 14 via a postal service, such as the U.S. Postal Service. The mail article 14 is addressed to a specified recipient. In step 104, the postal service retrieves the mail article 14 and delivers the article to a processing center (not shown). The method then moves on to step 106, where postal employees sterilize the mail article 14 by emitting an electron beam from the sterilization apparatus 12 on the mail articles. Next, in step 108, the postage stamp 52 affixed to the mail article 14 changes from one color to a color indicating exposure to the electron beam. For example, prior to exposure to the electron beam, the design and background sections may be colored green or white. After being exposed to the electron beam, the design and background sections may change to a red color.

Next, in step 110, it may be optionally determined by postal employees if the mail article has undergone sterilization by observing the color of the postage stamp 52. Step 110 may be used as a verification of sterilization of the mail prior to further processing and possible exposure to deadly microorganisms. If it is determined that the mail article has not undergone sterilization, as evidenced by the color of the postal stamp 52, the method then moves to step 106 where the mail article undergoes sterilization by the sterilization apparatus 12.

However, if it is determined that the mail article has undergone the sterilization process, the method moves from step 110 to step 112 where the mail article undergoes conventional processing, such as sorting and post marking of the mail article. Next, in step 114, the mail article is delivered to the specified recipient.

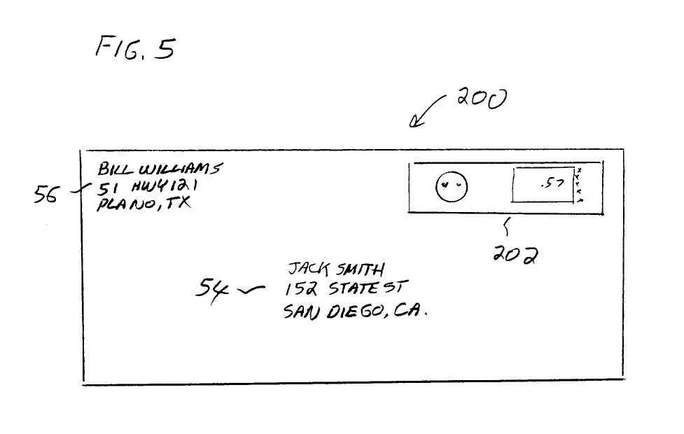

FIG. 5 is a front view of an envelop 200 having a metering mark 202 in an alternate embodiment of the present invention. The envelop 202 is similar to the mail article 14, with the exception that the envelop does not include a postage stamp. It is quite common for mail sent from larger companies or offices to utilize a postal machine which affixes an ink mark or metering mark to the mail article. The metering mark provides the postage date and postage amount necessary for mailing the envelop 200. The ink may be of a composition that changes color when exposed to radiation. When it is initially affixed to the envelop, the ink is one color (e.g., green). When the envelop is exposed to an electron beam produced from the sterilization apparatus 12, the metering mark changes color, such as red. The metering mark may be applied directly to a mail article or to an adhesive label which is affixed to the mail article. In a second alternate embodiment of the present invention, the adhesive label to which the metering mark is applied may change colors when exposed to an electron beam.

Although the preferred embodiment discusses the sterilization apparatus 12 utilizing an electron beam to sterilize the mail articles, any device providing sterilization of a plurality of mail articles may be employed. For example, the sterilization apparatus may employ gamma radiation or a gaseous substance such as ethylene oxide to sterilize the mail articles.

The system 10 and its associated method provide many advantages over existing postage stamps. With the use of a sterilization process to sterilize mail, the system 10 allows all persons handling mail, to readily determine if the mail articles have been properly undergone the sterilization process. Additionally, the postage stamp of the disclosed invention simultaneously is used as payment of the mail articles, as is currently done with conventional postage stamps. Thus, the present invention reduces the need for additional labels, or indicators to provide verification of successful completion of the sterilization process. The present invention also allows postal employees and recipients of mail to confidently handle properly processed mail.

It is thus believed that the operation and construction of the present invention will be apparent from the foregoing description. While the method and system shown and described have been characterized as being preferred, it will be readily apparent that various changes and modifications could be made therein without departing from the scope of the invention as defined in the following claims.

What is claimed is:

1. A system for indicating proper sterilization of a mail article, said system comprising:
a mail article;
a postage stamp affixed to said mail article, said postage stamp having a color indicator;
a sterilization apparatus having means for sterilizing said mail article;
whereby when said mail article is sterilized by the sterilization apparatus, said color indicator changes from a first color to a second color.

2. The system of claim 1 wherein the sterilization apparatus emits an electron beam to sterilize said mail article.

3. The system of claim 1 wherein the sterilization apparatus emits gamma radiation to sterilize said mail article.

4. The system of claim 1 wherein the color indicator is a design section on an outer surface of said postage stamp, said indicator indicating a first color prior to exposure to sterilization from said sterilization apparatus and the second color after exposure to sterilization from said sterilization apparatus.

5. The system of claim 1 wherein the color indicator is a background section on an outer surface of said stamp, said background indicating a first color prior to exposure to sterilization from said sterilization apparatus and the second color after exposure to sterilization from said sterilization apparatus.

6. The system of claim 1 wherein said postage stamp provides an indication of payment of postal fees for mailing said mail article.

7. The system of claim 1 wherein:
said sterilization apparatus emits an electron beam to sterilize said mail article;
said postage stamp provides an indication of payment of postal fees for mailing said mail article; and
said color indicator changes from the first color to the second color upon exposure to said electron beam.

8. The system of claim 1 wherein said postage stamp is a meter mark affixed to said mail article.

9. A method of indicating proper sterilization of a mail article by a sterilization apparatus, said method comprising the steps of:
affixing; by a sender of the mail article, a postage stamp to a mail article;
collecting, by a postal service, the mail article;
sterilizing, by the sterilization apparatus, the mail article;
transforming a portion of a postage stamp affixed to the mail article from a first color to a second color when the mail article successfully completes sterilization by the sterilization apparatus; and
delivering, by the postal service, the mail article to a specified recipient.

10. The method of indicating proper sterilization of a mail article of claim 9, further comprising, after the step of sterilizing, by the sterilization apparatus, the mail article, the step of:
determining, by the postage service, if the postage stamp has changed to the second color;
sterilizing, by the sterilization apparatus, the mail article if the postage stamp has not changed to the second color.

11. The method of indicating proper sterilization of a mail article of claim 9 wherein said step of sterilizing, by the sterilization apparatus, the mail article includes emitting an electron beam by the sterilization apparatus to sterilize the mail article.

12. A stamp for indicating proper sterilization of a mail article, said stamp comprising:
a sheet of material having an outer surface and a back surface;
said outer surface displaying a color indicator; and
said back surface having an adhesive for affixing said sheet of material to the mail article;
whereby said color indicator indicates a first color prior to being subjected to a sterilization process and indicating a second color after being subjected to a sterilization process.
